(19) United States
(12) Patent Application Publication
(10) Pub. No.: US 2013/0345152 A1
(43) Pub. Date: Dec. 26, 2013

(54) COMPOSITIONS COMPRISING RECOMBINANT COWPOX VIRUS PROTEIN CPXV014

(71) Applicants: Klaus Frueh, Portland, OR (US); David M. Edwards, Beaverton, OR (US); Ravi Iyer, Portland, OR (US); Dina Alzhanova, Beaverton, OR (US)

(72) Inventors: Klaus Frueh, Portland, OR (US); David M. Edwards, Beaverton, OR (US); Ravi Iyer, Portland, OR (US); Dina Alzhanova, Beaverton, OR (US)

(73) Assignee: OREGON HEALTH & SCIENCE UNIVERSITY, Portland, OR (US)

(21) Appl. No.: 13/922,850

(22) Filed: Jun. 20, 2013

Related U.S. Application Data
(60) Provisional application No. 61/662,143, filed on Jun. 20, 2012.

(51) Int. Cl. C07K 14/005

(52) U.S. Cl. CPC C07K 14/005
USPC 514/21.2; 435/320.1; 530/350; 435/375

(57) ABSTRACT

Disclosed herein are expression vectors that encode cowpox virus protein CPXV014 and homologs thereof that are useful in inhibiting CD3/CD28 mediated T cell stimulation. Further disclosed are polypeptide compositions comprising CPXV014 and homologs thereof as well as methods of inhibiting CD3/CD28 mediated T cell stimulation using the polypeptide compositions.

### ACKNOWLEDGEMENT OF GOVERNMENT SUPPORT

This invention was funded in part by the United States Government under the terms of grant number 5P51 RR000163-49 and grant number 1R21 A1077048-01, awarded by the National Institutes of Health. The government has certain rights to this invention.

### FIELD

The field is recombinant protein compositions. Specifically, the field is a recombinant form of the CPXV014 protein from cowpox virus.

### SUMMARY

Disclosed herein are compositions comprising the CPXV014 protein and modifications thereof that can be used to prevent CD3/CD28 mediated activation of T cells, particularly naïve T cells. The maturation of T cells from naïve to effector T cells is crucial in the development of T cell responses to both foreign and self-antigens. Treatment of T cells with recombinant CPXV014 will prevent de novo T cell responses.

Disclosed herein is a nucleic acid expression vector comprising a nucleic acid sequence that encodes a polypeptide of SEQ ID NO: 1 or a homolog thereof. The expression vector also comprises a promoter operably linked to the nucleic acid sequence. The expression vector may be provided in a transfected cell such as a CHO cell.

Disclosed herein is a recombinant polypeptide of SEQ ID NO: 1 or a homolog thereof. In some examples, the formulation further comprises features added to the recombinant protein. Examples of such features include an Fc tag and/or a thrombin cleavage site as exemplified by SEQ ID NO: 2.

Also disclosed is a method of inhibiting CD3/CD28 mediated stimulation of a T cell that involves contacting the T cell with a recombinant polypeptide of SEQ ID NO: 1 or a homolog thereof, wherein the polypeptide or homolog thereof inhibits CD3/CD28 mediated T cell stimulation. In some examples, the contacting occurs ex vivo. In other examples, the contacting occurs in vivo. In other examples the T cell is a CD8+ T cell. In others, it is a naïve T cell.

### BRIEF DESCRIPTION OF THE DRAWINGS

FIG. 1 shows the general schematic of anti-CD3 and anti-CD28 T cell activation experiments.

FIG. 2A is a set of flow cytometry plots showing the percentage of TNFα+, IFN-γ-T cells, gated for either CD4+ (top row) or CD8+ from mouse splenocytes incubated with A20 cells that were uninfected (left panels,) infected with vaccinia virus (center panels) or infected with cowpox virus (right panels.) Splenocytes were stimulated as described in Example 1 below. Data are typical results from an individual animal selected from each group.

FIG. 2B is a bar graph showing the aggregate data for stimulated mouse splenocytes treated as described in Example 1 below. Data are expressed as the percentage of TNFα+, IFNγ-T cells gated for either CD4+ or CD8+ and normalized to the results observed for T cells stimulated with vaccinia virus infected A20 cells. The results represent the mean for five mice per group with the error bars reflecting the standard deviation. The data indicate that pre-infecting cells with cowpox virus causes an inhibition of CD3 and CD28 mediated T cell activation.

FIG. 3 is a set of two graphs showing that cells infected with a cowpox virus mutant that comprises a deletion of genes CPX011-CPX016 (clone A530) is unable to inhibit anti-CD3 and anti-CD28 mediated T cell activation. Cells were treated as described in Example 1 below. The different deletion mutants and their corresponding clones are indicated in the legend.

FIG. 4A is a set of flow cytometry plots showing the percentage of TNFα+, IFNγ-T cells, gated for either CD4+ (top row) or CD8+ from mouse splenocytes incubated with supernatants of MC57 cells that were uninfected (first panel from the left,) infected with vaccinia virus (second panel from the left,) infected with cowpox virus (third panel from the left,) or infected with cowpox virus with a deletion of CPX011-CPX016 (last panel.) Splenocytes were stimulated as described in Example 3 below. Data are typical results from an individual animal selected from each group.

FIG. 4B is a bar graph showing the aggregate data for stimulated mouse splenocytes treated as described in Example 3 below. Data are expressed as the percentage of TNFα+, IFNγ-T cells gated for either CD4+ or CD8+ and normalized to the results observed for T cells stimulated with vaccinia virus infected A20 cells. The results represent the mean for five mice per group with the error bars reflecting the standard deviation.

FIG. 5 is a set of two bar graphs showing data indicating that cowpox virus with CPX015 deleted can still inhibit CD3/CD28 mediated T cell activation of both CD8+ and CD4+ T cells. Cells are treated as described in Example 4 below. Graphs depict the mean and SEM for each indicated group. Five mice were included per group.

FIG. 6 is a set of two bar graphs showing data indicating the cowpox virus with CPX014 deleted cannot inhibit CD3/CD28 mediated T cell activation of both CD4+ and CD8+ T cells.

FIG. 7 is an image of a western blot showing the expression of CPXV014 in CHO cells transfected with CPXV014 in an expression vector.

FIG. 8 is a bar graph showing that supernatants from CPXV014-Fc transfected CHO cells can inhibit CD3/CD28 mediated activation of CD4+ and CD8+ T cells in the absence of cowpox virus. Clones used are indicated in the legend.

FIG. 9 is a flow cytometry plot showing a sorted population comprising about 92% CD8+/CD4-T cells.

FIG. 10 is a bar graph showing the effect of CPXV and CPXV with a deletion of the CPXV014 locus on activation of the CD8+ T cell population exemplified by FIG. 9.

FIG. 11 is a set of two bar graphs depicting the effects of the indicated treatments on sorted CD8+ T cells. This figure shows that naïve CD8+ T cells treated with recombinant CPXV014 may still be stimulated later with PMA and ionomycin.

### SEQUENCE LISTING

SEQ ID NO: 1 is cowpox virus CPX014

SEQ ID NO: 2 is cowpox virus CPX014 with an Fc tag and a thrombin cleavage site

### DETAILED DESCRIPTION

### I. Terms

Administration: To provide or give a subject an agent by any effective route. Exemplary routes of administration include, but are not limited to, injection (such as subcutaneous, intramuscular, intradermal, intraperitoneal, and intravenous), oral, sublingual, rectal, transdermal, intranasal, vaginal and inhalation routes.

Carrier: refers to, for example, a diluent, adjuvant, preservative (e.g., Thimersol, benzyl alcohol), anti-oxidant (e.g., ascorbic acid, sodium metabisulfite), solubilizer (e.g., Tween 80, Polysorbate 80), emulsifier, buffer (e.g., Tris HCl, acetate, phosphate), water, aqueous solutions, oils, bulking substance (e.g., lactose, mannitol), excipient, auxilliary agent or vehicle with which an active agent of the present invention is administered. Suitable pharmaceutical carriers are described in “Remington's Pharmaceutical Sciences” by E.W. Martin (Mack Publishing Co., Easton, Pa.); Gennaro, A. R., Remington: The Science and Practice of Pharmacy, 20^{th} Edition, (Lippincott, Williams and Wilkins), 2000; Liberman, et al., Eds., Pharmaceutical Dosage Forms, Marcel Decker, New York, N.Y., 1980; and Kibbe, et al., Eds., Handbook of Pharmaceutical Excipients (3rd Ed.), American Pharmaceutical Association, Washington, 1999.

Conservative Variants: A substitution of an amino acid residue for another amino acid residue having similar biochemical properties. “Conservative” amino acid substitutions are those substitutions that do not substantially affect or decrease an activity of an MHC Class II polypeptide, such as an MHC class II α1 polypeptide. A polypeptide can include one or more amino acid substitutions, for example 1-10 conservative substitutions, 2-5 conservative substitutions, 4-9 conservative substitutions, such as 1, 2, 5 or 10 conservative substitutions. Specific, non-limiting examples of a conservative substitution include the following examples:

| Original | Conservative |
|---|---|
| Amino Acid | Substitutions |
| Ala | Ser |
| Arg | Lys |
| Asn | Gln, His |
| Asp | Glu |
| Cys | Ser |
| Gln | Asn |
| Glu | Asp |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Contacting: refers to placement in direct physical association, including both a solid and liquid form. Contacting can occur in vitro with isolated cells or tissue or in vivo by administering to a subject.

Effective amount: refers to an amount of therapeutic agent that is sufficient to generate a desired response, such as reduce or eliminate a sign or symptom of a condition or disease, such as an autoimmune disease like graft-versus-host disease. When administered to a subject, a dosage will generally be used that will achieve target tissue concentrations) that has been shown to achieve in vitro inhibition T cell activation. In some examples, an “effective amount” is one that treats (including prophylaxis) one or more symptoms and/or underlying causes of any of a disorder or disease. In other examples, an effective amount is an amount that prevents one or more signs or symptoms of a particular disease or condition from developing, such as one or more signs or symptoms associated with graft-versus-host disease.

Operably Linked: A first nucleic acid sequence is operably linked to a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

Promoter: Promoters are sequences of DNA near the 5′ end of a gene that act as a binding site for RNA polymerase, and from which transcription is initiated. A promoter includes necessary nucleic acid sequences near the start site of transcription, such as, in the case of a polymerase II type promoter, a TATA element. In one embodiment, a promoter includes an enhancer. In another embodiment, a promoter includes a repressor element. Promoters may be constitutively active, such as a promoter that is continuously active and is not subject to regulation by external signals or molecules. In some examples, a constitutive promoter is active such that expression of a sequence operably linked to the promoter is expressed ubiquitously (for example, in all cells of a tissue or in all cells of an organism and/or at all times in a single cell or organism, without regard to temporal or developmental stage).

An inducible promoter is a promoter that has activity that is increased (or that is de-repressed) by some change in the environment of the cell such as the addition of a particular agent to the cell media or a removal of a nutrient or other component from the media of the cell.

Polypeptide: Any chain of amino acids, regardless of length or posttranslational modification (such as glycosylation, methylation, ubiquitination, phosphorylation, or the like). In one embodiment, a polypeptide is a CPXV014 polypeptide. “Polypeptide” is used interchangeably with peptide or protein, and is used to refer to a polymer of amino acid residues. A “residue” refers to an amino acid or amino acid mimetic incorporated in a polypeptide by an amide bond or amide bond mimetic.

Pharmaceutically acceptable: indicates approval by a regulatory agency of the Federal or a state government or listed in the U.S. Pharmacopeia or other generally recognized pharmacopeia for use in animals, and more particularly in humans.

Recombinant: A recombinant nucleic acid or polypeptide is a composition of matter one that (a) comprises a nucleic acid or amino acid sequence or combination of nucleic acid or amino acid sequences that is not naturally occurring or (b) is a polypeptide or nucleic acid that is made using a vector comprising a non-naturally occurring sequence or an artificial combination of two or more segments of sequence. For example, a recombinant protein may be made in an expression vector comprising an artificial combination of segments of sequence. This artificial combination may be made by chemical synthesis or, more commonly, by the artificial manipulation of isolated segments of nucleic acids, e.g., by genetic engineering techniques.

Sequence identity/similarity/homology: The identity/similarity between two or more nucleic acid sequences, or two or more amino acid sequences, is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are.

Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations.

The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN is used to compare nucleic acid sequences, while BLASTP is used to compare amino acid sequences. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. The percent sequence identity is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100. For example, a nucleic acid sequence that has 1166 matches when aligned with a test sequence having 1154 nucleotides is 75.0 percent identical to the test sequence (116671554*100=75.0). The percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. The length value will always be an integer. In another example, a target sequence containing a 20-nucleotide region that aligns with 20 consecutive nucleotides from an identified sequence as follows contains a region that shares 75 percent sequence identity to that identified sequence (that is, 15720*100=75).

For comparisons of amino acid sequences of greater than about 30 amino acids, the Blast 2 sequences function is employed using the default BLOSUM62 matrix set to default parameters, (gap existence cost of 11, and a per residue gap cost 5 of 1). Homologs are typically characterized by possession of at least 70% sequence identity counted over the full-length alignment with an amino acid sequence using the NCBI Basic Blast 2.0, gapped blastp with databases such as the nr or swissprot database. Queries searched with the blastn program are filtered with DUST (Hancock and Armstrong, 1994, Comput. Appl. Biosci. 10:67-70). Other programs use SEG. In addition, a manual alignment can be performed. Proteins with even greater similarity will show increasing percentage identities when assessed by this method, such as at least about 75%, 80%, 85%, 90%, 95%, 98%, or 99% sequence identity to a protein.

When aligning short peptides (fewer than around 30 amino acids), the alignment is performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, or 99% sequence identity to a protein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

One indication that two nucleic acid molecules are closely related is that the two molecules hybridize to each other under stringent conditions, as described above. Nucleic acid sequences that do not show a high degree of identity may nevertheless encode identical or similar (conserved) amino acid sequences, due to the degeneracy of the genetic code. Changes in a nucleic acid sequence can be made using this degeneracy to produce multiple nucleic acid molecules that all encode substantially the same protein. An alternative (and not necessarily cumulative) indication that two nucleic acid sequences are substantially identical is that the polypeptide which the first nucleic acid encodes is immunologically cross reactive with the polypeptide encoded by the second nucleic acid.

One of skill in the art will appreciate that the particular sequence identity ranges are provided for guidance only; it is possible that strongly significant homologs could be obtained that fall outside the ranges provided.

Subject: A living multicellular vertebrate organism, a category that includes, for example, mammals and birds. A “mammal” includes both human and non-human mammals, such as mice. In some examples, a subject is a patient, such as a patient diagnosed with graft-versus-host disease or a patient in need of a bone marrow transplant.

Treat: refers to any type of treatment that imparts a benefit to a patient afflicted with a disease, including improvement in the condition of the patient (e.g., in one or more symptoms), delay in the progression of the condition, etc. Similarly, “treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, an improvement in the overall health or well-being of the subject, or by other clinical or physiological parameters associated with a particular disease. A “prophylactic” treatment is a treatment administered to a subject who does not exhibit signs of a disease or exhibits only early signs for the purpose of decreasing the risk of developing pathology.

Vector: A vector is a nucleic acid molecule that facilitates the insertion of foreign nucleic acid into a host cell genome without disrupting the ability of the vector to replicate and/or integrate in a host cell. A vector can include nucleic acid sequences that permit it to replicate in a host cell, such as an origin of replication. An insertional vector is capable of inserting itself into a host nucleic acid. A vector can also include one or more selectable marker genes and other genetic elements. An expression vector is a vector that further comprises the necessary regulatory sequences to allow transcription and translation of inserted gene or genes.

### II. Compositions

Disclosed herein are expression vectors for that facilitate the recombinant expression of CPXV014, recombinant CPXV014 polypeptides, and methods of using those compositions. CPXV014 is a viral protein derived from Cowpox virus that has the effect of transiently inhibiting the activation of naïve CD4 and CD8 T cells.

The expression vectors disclosed herein comprise a first nucleic acid sequence that encodes the SEQ ID NO: 1 polypeptide or a homolog thereof and a promoter operably linked to the first nucleic acid sequence such that the first nucleic acid sequence is transcribed into mRNA and then translated into the polypeptide when integrated into a host cell.

The polypeptide compositions disclosed herein comprise a recombinant CPXV014 or a homolog thereof, wherein the recombinant CPXV014 is or is homologous to SEQ ID NO: 1. Homologs of CPXV014 may be any conservative or other variant of SEQ ID NO: 1 that is shown to inhibit CD3/CD28 mediated T cell activation at about the same activity as SEQ ID NO: 1. Methods of preparing CPXV014 homologs and testing whether or not they inhibit CD3/CD28 mediated T cell activation are understood by those of skill in the art in light of this disclosure. The composition may also comprise an amino acid sequence that may be used as a protein tag. The polypeptide may further comprise an engineered protease cleavage site such that the tag may be removed from the expressed CPXV014.

Recombinant CPXV014 polypeptide or homologs thereof may be prepared in a variety of ways, according to methods well known in the art. For example, the protein may be purified from appropriate sources, e.g., transformed bacteria, cultured animal cells (such as Chinese Hamster Ovary, or CHO cells) or tissues, or animals (e.g., by immunoaffinity purification methods). The availability of nucleic acid molecules encoding CPXV014 enables production of the protein using in vitro expression methods and cell-free expression systems known in the art. In vitro transcription and translation systems are commercially available, e.g., from Promega Biotech (Madison, Wis.) or Gibco-BRL (Gaithersburg, Md.).

Alternatively, larger quantities of CPXV014 may be produced by expression in a suitable prokaryotic or eukaryotic system. For example, a nucleic acid sequence encoding CPXV014 may be inserted into an expression vector adapted for expression in a bacterial cell, such as E. coli. Such vectors comprise the regulatory elements necessary for expression of the DNA in the host cell positioned in such a manner as to permit expression of the DNA in the host cell. Such regulatory elements required for expression may include promoter sequences, transcription initiation sequences and, optionally, enhancer sequences.

CPXV014 produced by gene expression in a recombinant prokaryotic or eukaryotic system may be purified according to methods known in the art. A commercially available expression/secretion system can be used, whereby the recombinant protein is expressed and secreted from the host cell, and readily purified from the surrounding medium by any method known in the art. For example, the recombinant protein may be purified by affinity separation, such as by immunological interaction with antibodies that bind specifically to the recombinant protein or nickel columns for isolation of recombinant proteins tagged with 6-8 histidine residues at their N-terminus or C-terminus. Alternative tags may comprise the FLAG epitope, the hemagglutinin epitope, or a human antibody constant region (Fc). Such methods are commonly used by skilled practitioners.

Recombinant CPXV014 polypeptides, prepared by the aforementioned methods, may be analyzed according to standard procedures. For example, such protein may be subjected to amino acid sequence analysis, according to known methods. Recombinant CPXV014 polypeptides or homologs thereof may be further analyzed for inhibition of CD3/CD28 mediated T-cell stimulation by any method known in the art. Several of such methods are described in the Examples below.

Exemplary amino acid sequences of CPXV014 are known (see NCBI Reference Seqeunce NP_{—}619803.1, incorporated by reference herein). A CPXV014 amino acid sequence may have 75%, 80%, 85%, 90%, 95%, 97% or 99% identity or homology with this sequence. The contemplated homologs inhibit CD3/CD28 mediated T cell activation at about the same efficiency as SEQ ID NO: 1.

CPXV014 may be provided in a composition with a pharmaceutically acceptable carrier. For example, CPXV014 may be formulated with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of CPXV014 in the chosen medium may be varied and the medium may be chosen based on the desired route of administration of the pharmaceutical preparation. Except insofar as any conventional media or agent is incompatible with CPXV014, its use in the pharmaceutical preparation is contemplated.

Selection of a suitable pharmaceutical preparation will also depend upon the mode of administration chosen. For example, CPXV014 may be administered by direct injection into an area proximal to the infection. In this instance, a pharmaceutical preparation comprises the CPXV014 dispersed in a medium that is compatible with the site of injection. CPXV014 may be administered by any method such as intravenous injection into the blood stream, oral administration, or by subcutaneous, intramuscular or intraperitoneal injection. Pharmaceutical preparations for injection are known in the art. If injection is selected as a method for administering CPXV014, steps must be taken to ensure that sufficient amounts of the molecules reach their target cells to exert a biological effect.

Pharmaceutical compositions containing CPXV014 as the active ingredient in intimate admixture with a pharmaceutically acceptable carrier can be prepared according to conventional pharmaceutical compounding techniques. The carrier may take a wide variety of forms depending on the form of preparation desired for administration, e.g., intravenous, oral, direct injection, intracranial, and intravitreal.

A pharmaceutical preparation of the invention may be formulated in dosage unit form for ease of administration and uniformity of dosage. Dosage unit form, as used herein, refers to a physically discrete unit of the pharmaceutical preparation appropriate for the patient undergoing treatment. Each dosage should contain a quantity of active ingredient calculated to produce the desired effect in association with the selected pharmaceutical carrier. Procedures for determining the appropriate dosage unit are well known to those skilled in the art. Dosage units may be proportionately increased or decreased based on the weight of the patient. Appropriate concentrations for alleviation of a particular pathological condition may be determined by dosage concentration curve calculations, as known in the art.

The effective amount for the administration of CPXV014 may be determined by evaluating the toxicity of CPXV014 in animal models. Various concentrations of CPXV014 in pharmaceutical preparations may be administered to mice, and the minimal and maximal dosages may be determined based on the beneficial results and side effects observed as a result of the treatment. The effective amount may also be determined by assessing the efficacy of CPXV014 treatment in combination with other standard drugs, including other drugs used in the treatment of graft versus host disease. The dosage units of CPXV014 may be determined individually or in combination with each treatment according to the effect detected.

Pharmaceutical compositions comprising CPXV014 may include one or more additional compositions useful in the treatment of graft-versus-host disease such as cyclosporine, tacrolimus, monoclonal antibodies that block IL-2, or antibodies that block IL-2 receptor.

The pharmaceutical compositions comprising CPXV014 can be delivered in a controlled release system, such as using an intravenous infusion, an implantable osmotic pump (e.g., a subcutaneous pump), a transdermal patch, liposomes, or other modes of administration. In a particular embodiment, a pump may be used (see Langer (Science (1990) 249:1527-1533); Sefton, CRC Crit. Ref. Biomed. Eng. (1987) 14:201; Buchwald et al., Surgery (1980) 88:507; Saudek et al., N. Engl. J. Med. (1989) 321:574). In another embodiment, polymeric materials may be employed (see Medical Applications of Controlled Release, Langer and Wise (eds.), CRC Press: Boca Raton, Fla. (1974); Controlled Drug Bioavailability, Drug Product Design and Performance, Smolen and Ball (eds.), Wiley: New York (1984); Ranger and Peppas, J. Macromol. Sci. Rev. Macromol. Chem. (1983) 23:61; see also Levy et al., Science (1985) 228:190; During et al., Ann. Neurol. (1989) 5 25:351; Howard et al., J. Neurosurg. (1989) 71:105). In yet another embodiment, a controlled release system can be placed in proximity of the target tissues of the animal, thus requiring only a fraction of the systemic dose (see, e.g., Goodson, in Medical Applications of Controlled Release, supra, (1984) vol. 2, pp. 115-138). In particular, a controlled release device can be introduced into an animal in proximity to the desired site. Other controlled release systems are discussed in the review by Langer (Science (1990) 249:1527-1533).

### III. Methods of Use

Disclosed herein are methods of inhibiting the CD3/CD28 mediated activation of a T cell comprising contacting the T cell with a recombinantly produced CPX014 or a homolog thereof. The contacting occurs may occur ex vivo or in vivo. The T cell may be any T cell, including a CD8+ T cell or a naïve T cell.

Another method comprises treating a subject with a pharmaceutical composition comprising recombinant CPX014. In some aspects, the subject is a human patient. In additional aspects, an effective amount of the CPX014 is administered to the subject in order to treat a disease characterized by aberrant CD3/CD28 mediated T cell activation.

### EXAMPLES

The following examples are illustrative of disclosed methods. In light of this disclosure, those of skill in the art will recognize that variations of these examples and other examples of the disclosed method would be possible without undue experimentation.

### Example 1

### Antigen Presenting Cells Infected with Cowpox Virus Inhibit CD3/CD28 Mediated T Cell Stimulation

The mouse B cell lymphoma cell line A20 was infected with Vaccinia virus (VACV) or Cowpox virus (CPXV) at a multiplicity of infection (M.O.I.) of 5 for 16 hours. The following day, splenocytes from Specific Pathogen Free (SPF) Balb/cByJ mice (5 weeks old, age and sex matched) were co-incubated with CPXV-infected A20 cells, which had been washed 3 times with phosphate-buffered saline (PBS) to remove residual virus.

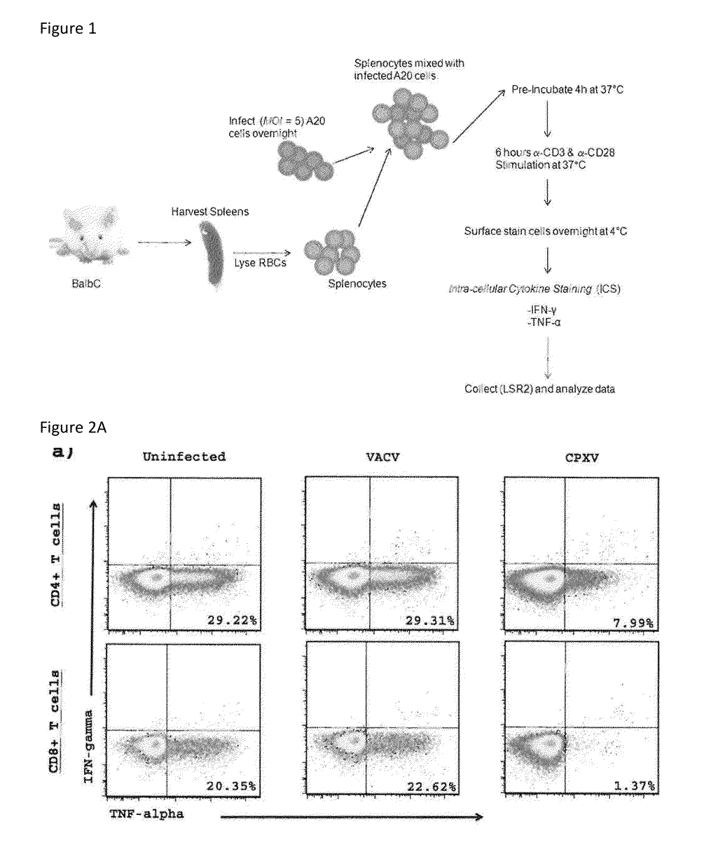

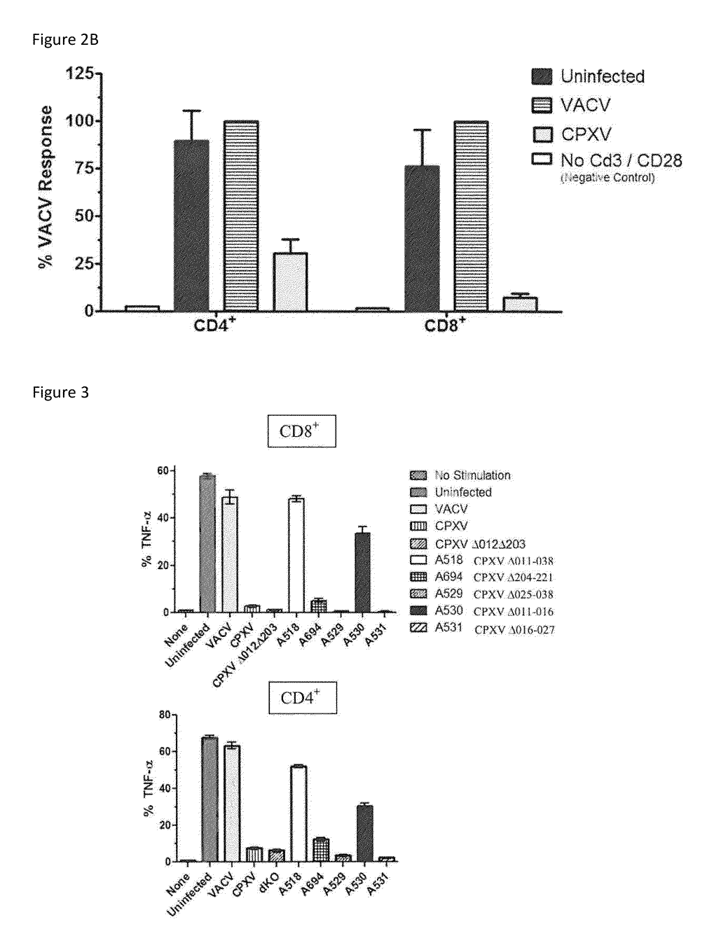

Following the 4 hour co-incubation all samples were transferred to anti-CD3 and anti-CD28 antibody coated plates in the presence of Brefeldin A (BFA) for 6 hours. The combination of anti-CD3 and anti-CD28 is a well-known stimulus for naive T cells that closely mimics the natural T cell activation by engagement of the CD3-associated T cell receptor with MHC/peptide complexes and co-stimulation via CD28. Activation of naive T cells results in production of antiviral cytokines IFNγ and TNFα; BFA serves to trap these cytokines inside the T cells to facilitate detection by intracellular cytokine staining (ICCS). Briefly, cells were washed 3 times with PBS +5% FBS. The cells were then fixed and permeabilized using BD Cytofix/Cytoperm (Cat#51-2090KZ) at 4° C. for 30 minutes. Cells were then washed 2 times with BD PermWash buffer (Cat#51-2091KZ), centrifuging at 1200 RPM, 4° C., 5 minutes between washes. Cells were then resuspended in PermWash buffer containing fluorochrome labeled antibodies against TNFα and IFNγ, as well as a polyclonal, HRP-tagged, rabbit anti-VACV antibody (that cross-reacts with cowpox virus) to monitor any infection of the splenocytes. The cells were incubated with these antibodies for 1 hr at 4° C. The cells were then washed 3 times with Permwash prior to incubation with streptavidin conjugated to a fluorochrome at 4° C. for 30 minutes. The cells were washed 3 times and then resuspended in PermWash prior to analysis on a BD LSR2 FACS. FIG. 1 shows a general schematic of the T cell assay. FIG. 2 shows that CPXV and not VACV inhibits anti-CD3 and anti-CD28 mediated activation of mouse T cells.

### Example 2

### A Protein Encoded by a Gene in the CPXV011-CPXV016 Region is Involved In the Inhibition of CD3/CD28 Mediated T Cell Stimulation by Cowpox Virus

The CPXV genome was then mapped in order to determine the protein responsible for the T cell inhibition. FIG. 3 shows that CPXV lacking the gene region CPXV011-CPXV016 cannot inhibit T cell activation.

### Example 3

### The Protein Involved in the Inhibition of CD3/CD28 Mediated T Cell Stimulation by Cowpox Virus is a Secreted Protein

Supernatants from MC57 cells infected at an M.O.I.=2 were ultra-centrifuged at 18,000 rpm for 80 minutes at 4° C. to remove virus. SPF Balb/CByJ mice (6 weeks old, age and sex matched) splenocytes were pretreated with four hours with a 50/50 ratio of supernatant to media prior to transfer to a new plate for stimulation with CD3/CD28 and BFA followed by ICCS. Data were collected on a Becton Dickinson LSR2 and then analyzed with FloJo Software (TreeStar.)

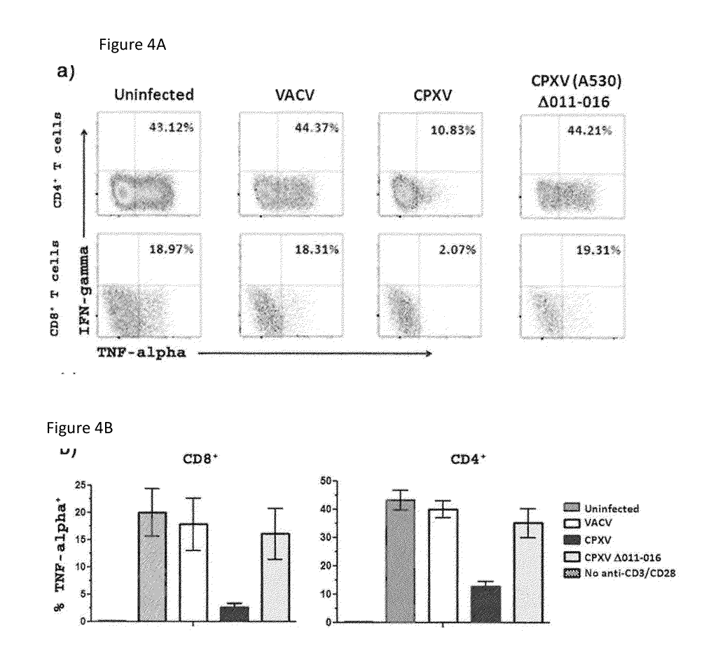

FIG. 4 shows that supernatants from CPXV infected cells do inhibit T cell activation; however supernatants from CPXV lacking the CPXV011-CPXV016 region do not inhibit T cell activation. This indicates that a secreted protein encoded in the CPXV011-CPXV016 region is responsible for the inhibition.

### Example 4

### The Protein Involved in the Inhibition of CD3/CD28 Mediated T Cell Stimulation by Cowpox Virus is CPXV014

Mouse A20 (B cell lymphoma cell line) cells were infected with Vaccinia virus, Cowpox virus (CPXV, Brighton Red strain), or the indicated CPXV deletion mutant at a multiplicity of infection (M.O.I.) of 5 for 16 hours. The following day splenocytes from Specific Pathogen Free (SPF) mice (Balb/cByJ, 5 weeks old, age and sex matched) were co-incubated with indicated infected A20 cells, which had been washed 3 times with PBS to remove residual virus.

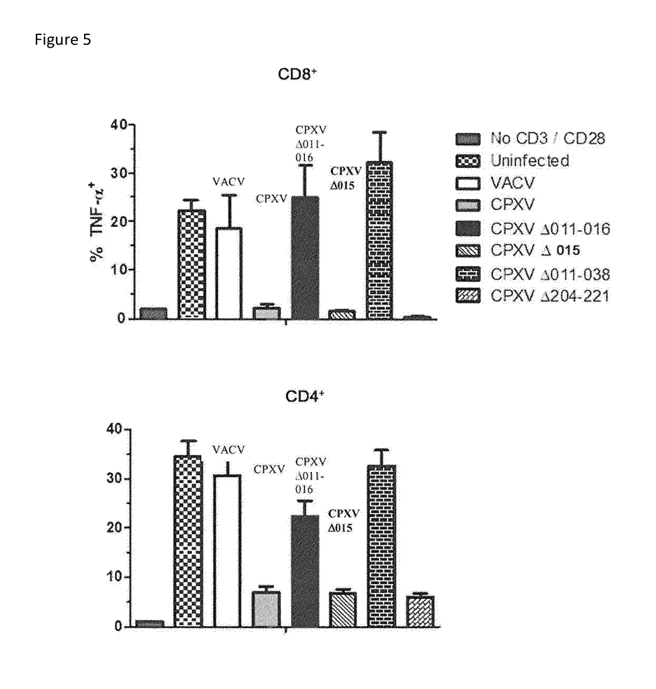

Following the 4 hour co-incubation, all samples were transferred to anti-CD3 and anti-CD28 antibody coated plates in the presence of BFA for 6 hours. Next, cells were surface stained with antibodies overnight followed by ICCS analysis the next day. Data was collected on a Becton Dickinson LSR2 and then analyzed with FloJo software (TreeStar). Of the genes in CPXV011-CPV016, only CPXV014 and CPXV015 encode secreted proteins. FIG. 5 shows that CPXV with a deletion of CPXV015 still inhibited T cell activation.

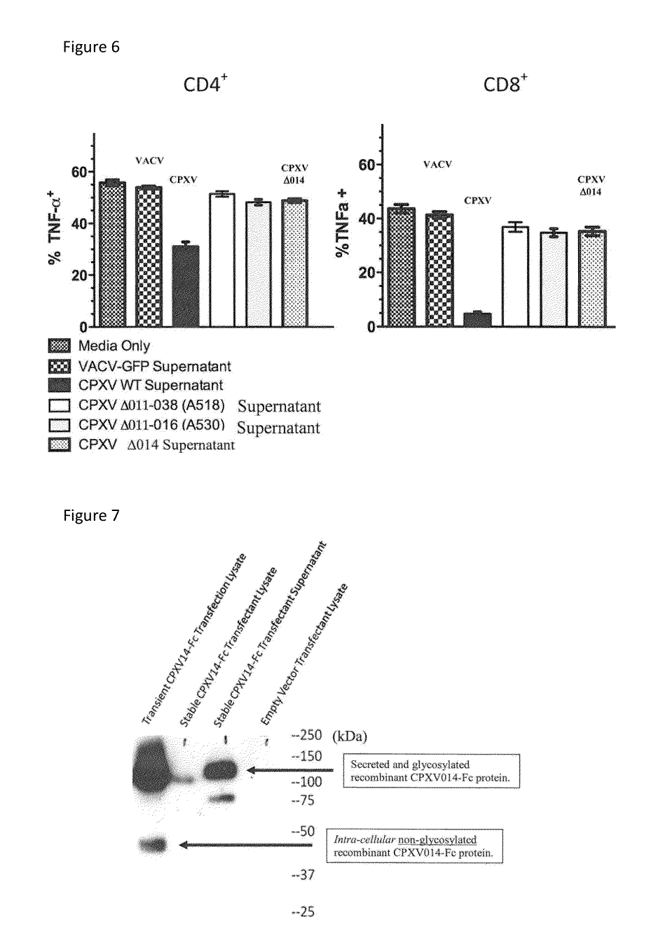

In FIG. 6, mouse MC57 cells were infected (M.O.I.=2) overnight with VACV, CPXV, CPXVΔ011-016 (a deletion of the sequence encoding the CPXV011 through CPXV016 genes), or CPXVΔ014 (deletion of CPXV014 alone.) Supernatants were harvested and virus was removed by ultracentrifugation (18,000 rpm, 80 minutes, at 4° C.). SPF Balb/CByJ mice (6 weeks old, age and sex matched) splenocytes were pre-treated for 4 hours with a 50/50 ratio of supernatants to media prior to transfer to a new plate for stimulation with plate bound anti-CD3 and anti-CD28 antibodies in the presence of BFA for 6 hours. Cells were surface stained with antibodies overnight followed by ICCS the next day. Data was collected on a Becton Dickinson LSR2 and then analyzed with FloJo software (TreeStar). FIG. 6 shows that CPXV with a deletion of CPXV014 are not able to inhibit T cell activation. Therefore, CPXV014 is required for T cell inhibition by CPXV.

### Example 5

### Recombinant CPXV014 Protein Inhibits CD3/CD28 Mediated T Cell Stimulation without Cowpox Viral Infection

A nucleic acid encoding CPXV014 (SEQ ID NO: 1) was cloned into the mammalian expression vector pcDNA3.1. A human IgG Fc tag was added to the C-terminus of CPXV014 in order to facilitate purification via affinity chromatography. In addition, a thrombin cleavage site was added between the Fc tag and the C terminus of CPXV014 in order to facilitate removal of the Fc tag. The entire construct therefore encodes an amino acid corresponding to SEQ ID NO: 2 herein. This expression plasmid was then stably transfected into Chinese Hamster Ovary (CHO) cells. FIG. 7 shows a Western blot analysis of supernatants and lysates of recombinant CPXV014-Fc protein transfected CHO cells. Supernatants and lysates from both transiently and stably transfected CHO cells underwent western blot analysis with goat anti-human Fc antibody (1:10,000 dilution). The expected protein size before glycosylation is predicted to be 49 kDa. Lysates from CHO cells transfected with empty pcDNA3.1 vector were included as a negative control. The Western Blot confirmed that the recombinant CPXV014-Fc protein was expressed.

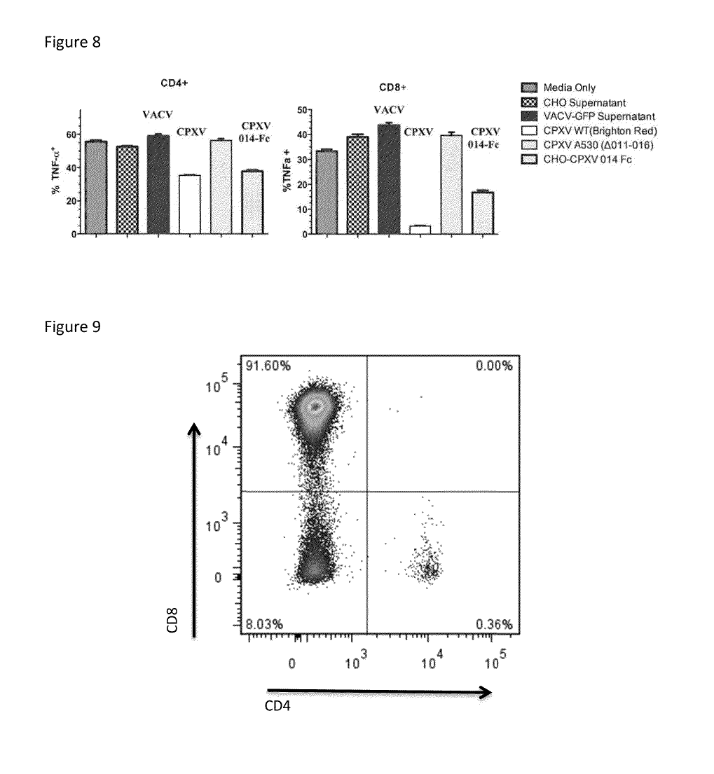

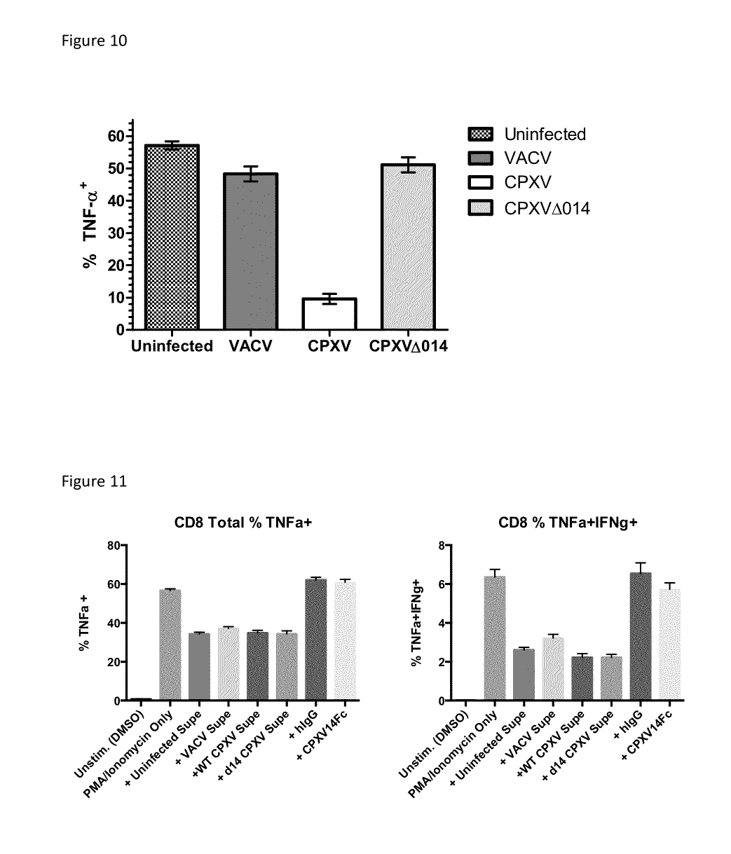

In FIG. 8, supernatants were harvested from either untransfected (“CHO supernatant”), CPXV014-Fc transfected CHO cells (“CHO-CPXV014-Fc”), or from mouse MC57 cells that were infected (M.O.I.=2) overnight with VACV, CPXV or CPXV Δ011-016 (gene deletion mutant). Supernatants were harvested and virus was removed by ultracentrifugation (18,000 rpm, 80 minutes, at 4° C.). SPF Balb/CByJ mice (6 weeks old, age and sex matched) splenocytes were pre-treated for 4 hours with a 50/50 ratio of supernatants to media prior to transfer to a new plate for stimulation with plate bound anti-CD3 and anti-CD28 antibodies in the presence of BFA for 6 hours. Cells were surface stained with antibodies overnight followed by ICCS (intracellular cytokine staining) the next day. Data was collected on a Becton Dickinson LSR2 and then analyzed with FloJo software (TreeStar).

FIG. 8 shows the results of stimulation with supernatants from CPXV014-Fc transfected CHO cells and empty vector CHO transfectants, as well as virus-free supernatants harvested from overnight-infected MC57 cells. The data indicate that recombinant CPXV014 inhibits T cell activation in the absence of viral infection. In particular, CD8+ T cells are affected by treatment with CPXV014-Fc treatment.

### Example 6

### Inhibition of CD3/CD28 Mediated Stimulation of T Cells by CPXV014 May be Reversed

FIG. 11 shows that CD8+ cells that have had CD3/CD28 mediated stimulation inhibited by CPXV14Fc (far right bars) may be restimulated with PMA and ionomycin to the same level as that seen in cells that were not stimulation-inhibited (compare to PMA/ionomycin only).

1. An expression vector comprising:
a first nucleic acid sequence that encodes a polypeptide of SEQ ID NO: 1 or a homolog thereof; and
a promoter operably linked to the nucleic acid sequence.

2. The expression vector of claim 1 wherein the expression vector is provided in a transfected cell.

3. The expression vector of claim 2 wherein the transfected cell is a stably transfected cell.

4. The expression vector of claim 2 wherein the transfected cell is a CHO cell.

5. A polypeptide composition comprising:
a recombinant polypeptide of SEQ ID NO: 1 or a homolog thereof.

6. The formulation of claim 5 wherein the recombinant polypeptide further comprises a protein tag.

7. The formulation of claim 2 comprising a recombinant polypeptide of SEQ ID NO: 2.

8. The formulation of claim 1 further comprising a pharmaceutically acceptable carrier.

9. A method of inhibiting CD3/CD28 mediated stimulation of a T cell, the method comprising:
contacting the T cell with a recombinant polypeptide of SEQ ID NO: 1 or a homolog thereof.

10. The method of claim 9 wherein the contacting occurs ex vivo.

11. The method of claim 9 wherein the contacting occurs in vivo.

12. The method of claim 9 wherein the T cell is a CD8⁺ T cell.

13. The method of claim 9 wherein the T cell is a naïve T cell.


SEQUENCE LISTING (2 sequences)

SEQ ID NO: 1 - protein, 202 aa, Cowpox virus
MINININTILIFASLFVASFANDYPPPGFFEDKYITNTFNYISIDFELYPVNVSSCNRLSTKQSSDVISTSELTITVNSTDCDPVFVTEYYSVKDKTAIAGLFTDTTKKQNTSKMCTLNIEVKCNAETEPVLIGNFTRVPEKASTHAENFTLIGNCLSDLHLYIAYVNTDEEFEEDTATVHIGNKLDINGIPPNMCATRTIN

SEQ ID NO: 2 - protein, 438 aa, Artificial Sequence
  Tagged Cowpox Virus
MINININTILIFASLFVASFANDYPPPGFFEDKYITNTFNYISIDFELYPVNVSSCNRLSTKQSSDVISTSELTITVNSTDCDPVFVTEYYSVKDKTAIAGLFTDTTKKQNTSKMCTLNIEVKCNAETEPVLIGNFTRVPEKASTHAENFTLIGNCLSDLHLYIAYVNTDEEFEEDTATVHIGNKLDINGIPPNMCATRTINLVPRGSQVTDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK